(19) United States
(12) Patent Application Publication
(10) Pub. No.: US 2005/0113343 A1
(43) Pub. Date: May 26, 2005

(54) Solid-state form of alendronate sodium and preparation thereof

(75) Inventors: Barbara Sienkiewicz, Krakow (PL); Piotr Kowalski, Krakow (PL); Katarzyna Ossowska, Wegrzce (PL); Jerzy Potok, Krakow (PL); Biserka Cetina-Cizmek, Zagreb (HR); Michaela Horvat, Sesvete (HR); Petar Tudja, Zagreb (HR); Aleksandar Danilovski, Rijeka (HR)

Correspondence Address: DARBY & DARBY P.C., P. O. BOX 5257, NEW YORK, NY, 10150-5257, US

(73) Assignee: PLIVA - Research and Development Ltd., Zagreb (HR)

(21) Appl. No.: 10/964,406

(22) Filed: Oct. 13, 2004

Related U.S. Application Data
(60) Provisional application No. 60/511,376, filed on Oct. 14, 2003.

(51) Int. Cl. C07F 9/28; A61K 31/66

(52) U.S. Cl. 514/102; 562/13

(57) ABSTRACT

A new solid-state form of alendronate sodium and process for its preparation are provided. The disclosure is also directed to pharmaceutical compositions containing the solid-state form, and to methods of treatment using the solid-state form.

Under 35 U.S.C. § 119(e), this application claims the benefit of prior U.S. Provisional Application No. 60/511,376, filed Oct. 14, 2003, which is incorporated herein by reference in its entirety.

### FIELD OF THE INVENTION

The present invention relates to a new solid-state form of alendronate sodium, and to a process for its preparation. The invention is also directed to pharmaceutical compositions containing the solid-state form, and to methods of treatment using the solid-state form.

### BACKGROUND OF THE INVENTION

Alendronate sodium is chemically described as 4-amino-1-hydroxybutylidene-1,1-bisphosphonic acid monosodium salt trihydrate. The structure of alendronate sodium is shown in Formula I.

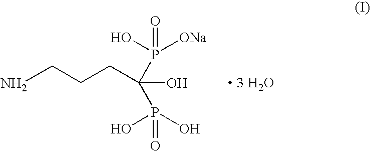

A variety of disorders in humans and other mammals involve or are associated with abnormal bone resorption. Such disorders include, but are not limited to, osteoporosis, Paget's disease, periprosthetic bone loss or osteolysis, and hypercalcemia of malignancy. The most common of these disorders is osteoporosis, which in its most frequent manifestation occurs in postmenopausal women. Osteoporosis is a systemic skeletal disease characterized by a low bone mass and microarchitectural deterioration of bone tissue, with a consequent increase in bone fragility and susceptibility to fracture.

Multinucleated cells called osteoclasts are responsible for causing bone loss through a process known as bone resorption. Bisphosphonates are selective inhibitors of osteoclastic bone resorption, making these compounds important therapeutic agents in the treatment or prevention of a variety of generalized or localized bone disorders caused by or associated with abnormal bone resorption. See H. Fleisch, Bisphosphonates In Bone Disease, From The Laboratory To The Patient, 2nd Edition, Parthenon Publishing (1995).

Alendronate sodium acts as a specific inhibitor of osteoclast-mediated bone resorption and is used in the treatment of osteoporosis and Paget's disease.

Several methods for the preparation of alendronate sodium have been described. In all cases, the processes described involve reacting 4-amino-1-hydroxy-butylidene-1,1-bisphosphonic acid with a base, such as sodium hydroxide, sodium carbonate or sodium bicarbonate.

U.S. Pat. Nos. 4,922,007 and 5,019,651 describe a process for the preparation of alendronate sodium that involves adding a 50% aqueous solution of sodium hydroxide (NaOH) to 4-amino-1-hydroxybutylidene-1,1-bisphosphonic acid at 20-25° C., while adjusting the pH to 4.3. International Publication No. WO 01/10874 describes a similar process that utilizes a 40% aqueous solution of NaOH.

U.S. Pat. No. 5,039,819 describes a process whereby 4-amino-1-hydroxybutylidene-1,1-bisphosphonic acid monosodium salt trihydrate is obtained by titration of 4-amino-1-hydroxybutylidene-1,1-bisphosphonic acid to pH 4.3 to 4.4, by gradual addition of a 5 N sodium hydroxide solution.

International Publication No. WO 95/06052 describes the preparation of alendronate sodium by a process that involves reaction of 4-amino-1-hydroxybutylidene-1,1-bisphosphonic acid with aqueous base of the formula MOH, such as sodium hydroxide, or of the formula MHCO₃ or MCO₃, such as sodium bicarbonate or sodium carbonate, wherein M is any ion.

It has now been surprisingly found that a new form of alendronate sodium can be obtained in high yield and purity, by a process that involves reaction of 4-amino-1-hydroxybutylidene-1,1-bisphosphonic acid with a strong acid cation exchange resin. The use of a strong acid cation exchange resin allows for an efficient and simple method of ion exchange, especially suitable for industrial applications, and does not endanger the environment.

### SUMMARY OF THE INVENTION

The present invention is directed, in part, to a new solid-state form of 4-amino-1-hydroxybutylidene-1,1-bisphosphonato hexacoordinated octahedral sodium monoaquo complex dihydrate, Form N, and to a process for preparing the new solid-state Form N by reaction of of 4-amino-1-hydroxybutylidene-1,1-bisphosphonic acid with a strong acid cation exchange resin.

Another embodiment of this invention involves pharmaceutical compositions containing the new solid-state form of 4-amino-1-hydroxybutylidene-1,1-bisphosphonato hexacoordinated octahedral sodium monoaquo complex dihydrate, Form N.

Further embodiments provide methods of inhibiting bone resorption, treating or preventing osteoporosis and treating Paget's disease by administering to a patient in need of such treatment a therapeutically effective amount of a pharmaceutical composition containing the new solid-state form of 4-amino-1-hydroxybutylidene-1,1-bisphosphonato hexacoordinated octahedral sodium monoaquo complex dihydrate, Form N, of the present invention.

### BRIEF DESCRIPTION OF THE DRAWINGS

FIG. 1 is a DSC thermogram of the solvent-free solid-state form of 4-amino-1-hydroxybutylidene-1,1-bisphosphonato hexacoordinated octahedral sodium monoaquo complex dihydrate, Form N, at 10° C./min.

FIG. 2 is a DSC thermogram of the solvent-free solid-state form of 4-amino-1-hydroxybutylidene-1,1-bisphosphonato hexacoordinated octahedral sodium monoaquo complex dihydrate, Form N, at 1° C./min.

FIG. 3 is a TGA scan of the solvent-free solid-state form of 4-amino-1-hydroxybutylidene-1,1-bisphosphonato hexacoordinated octahedral sodium monoaquo complex dihydrate, Form N, at 10° C./min.

FIG. 4 is an NIR spectrum of the solvent-free solid-state form of 4-amino-1-hydroxybutylidene-1,1-bisphosphonato hexacoordinated octahedral sodium monoaquo complex dihydrate, Form N.

### DETAILED DESCRIPTION OF THE INVENTION

Definitions

Unless defined otherwise, all technical and scientific terms used herein generally have the same meaning as commonly understood by one of ordinary skill in the art to which this invention belongs.

The term “therapeutically effective amount” as used herein means that amount of the solid state Form N of alendronate sodium of the present invention that will elicit the desired therapeutic effect or response when administered in accordance with the desired treatment regimen. An example of a therapeutically effective amount of the Form N alendronate sodium is a bone resorption inhibiting amount.

The term “bone resorption inhibiting” as used herein means treating or preventing bone resorption by the direct or indirect alteration of osteoclast formation or activity. Inhibition of bone resorption refers to treatment or prevention of bone loss, especially the inhibition of removal of existing bone either from the mineral phase and/or the organic matrix phase, through direct or indirect alteration of osteoclast formation or activity.

The term “about” or “approximately” as used herein means within an acceptable error range for the particular value as determined by one of ordinary skill in the art, which will depend in part on how the value is measured or determined, i.e., the limitations of the measurement system. For example, “about” can mean within 1 or more than 1 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

The phrase “pharmaceutically acceptable” as used herein refers to molecular entities and compositions that are “generally regarded as safe”, e.g., that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans.

### Solid State Form N of Alendronate Sodium

Characterization of the New Solid-State Form of 4-amino-1-hydroxybutylidene-1,1-bisphosphonato Hexacoordinated Octahedral Sodium Monoaquo Complex Dihydrate (Form N)

The new solid-state form of solvent-free 4-amino-1-hydroxybutylidene-1,1-bisphosphonato hexacoordinated octahedral sodium monoaquo complex dihydrate (Form N) prepared according to Examples 1 and 2 of the present invention, has a characteristic X-ray powder pattern, as obtained by X-ray diffraction on a powder sample of the solvent-free 4-amino-1-hydroxybutylidene-1,1-bisphosphonato hexacoordinated octahedral sodium monoaquo complex dihydrate. X-ray powder patterns were collected with a Panalytical X'PertPRO powder diffractometer using CuKα radiation.

The new solid-state form of solvent-free 4-amino-1-hydroxybutylidene-1,1-bisphosphonato hexacoordinated octahedral sodium monoaquo complex dihydrate (Form N) has characteristic x-ray powder diffraction peaks, designated by “2Θ” and expressed in degrees, as follows: 9.2±0.2°, 12.4±0.2°, 13.5±0.2°, 17.1±0.2°, 18.5±0.2°, 21.0±0.2°, 24.9±0.2°, 26.1±0.2° and 29.9±0.2°.

In addition, single crystals of the new solid-state form (Form N) were prepared and X-ray diffraction data were collected with a Bruker Nonius FR591/KappaCCD diffractometer using CuKα radiation. Basic crystallographic data for the new solid-state form of a solvent-free 4-amino-1-hydroxybutylidene-1,1-bisphosphonato hexacoordinated octahedral sodium monoaquo complex dihydrate (Form N) are set forth in Table 1.

Differential scanning calorimetry (DSC) was carried out using a Perkin Elmer apparatus Pyris 1. Thermogravimetric analysis (TGA) was carried out on a Perkin Elmer TGA 7 instrument.

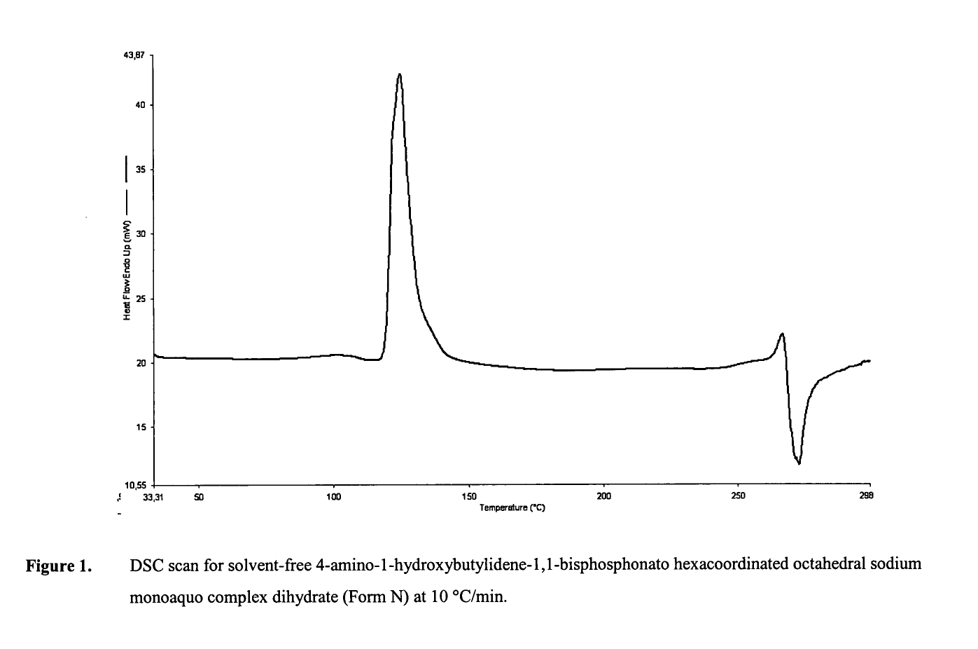

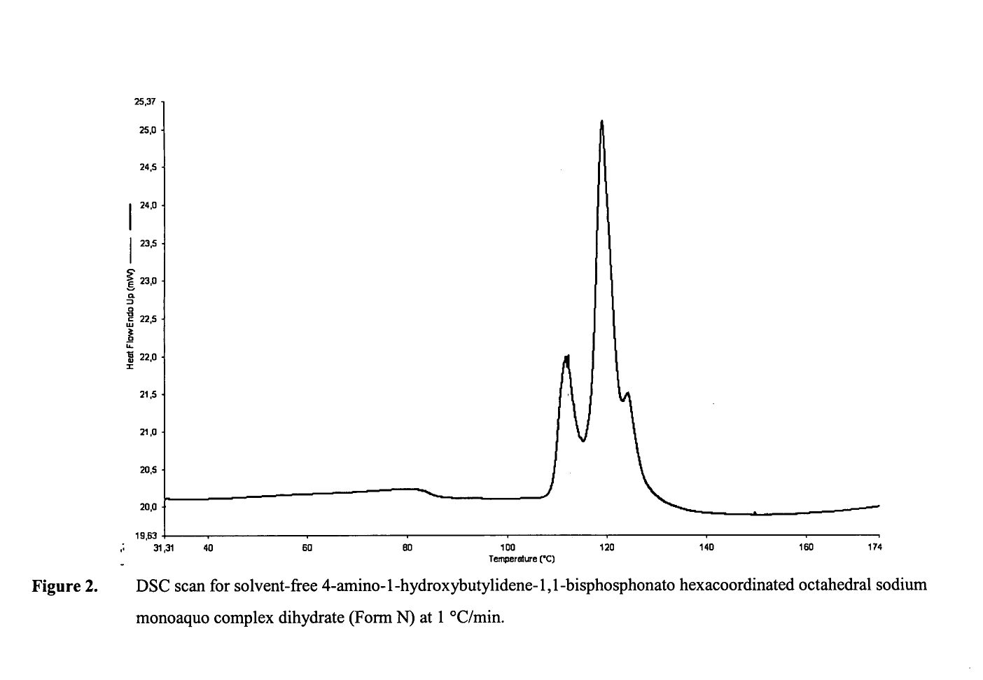

DSC curves for the solvent-free 4-amino-1-hydroxybutylidene-1,1-bisphosphonato hexacoordinated octahedral sodium monoaquo complex dihydrate (Form N), scanned at a heating rate of 10° C./min (FIG. 1), show a broad endothermic peak in the temperature region from 76° C. to 115° C., with the onset temperature at 82.9° C. (ΔH=14.1 J/g) corresponding to loss of adsorbed water. It is followed by an irregular endothermic peak with shoulders at 119.5° C. (ΔH=455.5 J/g) that corresponds to loss of coordinated and crystal water. The third endothermic peak, corresponding to melting of Form N, is at 261.6° C. (ΔH=49.2 J/g) and is followed by degradation. When the DSC curve was scanned at a heating rate of 1° C./min, the endothermic peak that corresponds to loss of coordinated and crystal water becomes more resolved, as shown in FIG. 2, confirming loss of three water molecules.

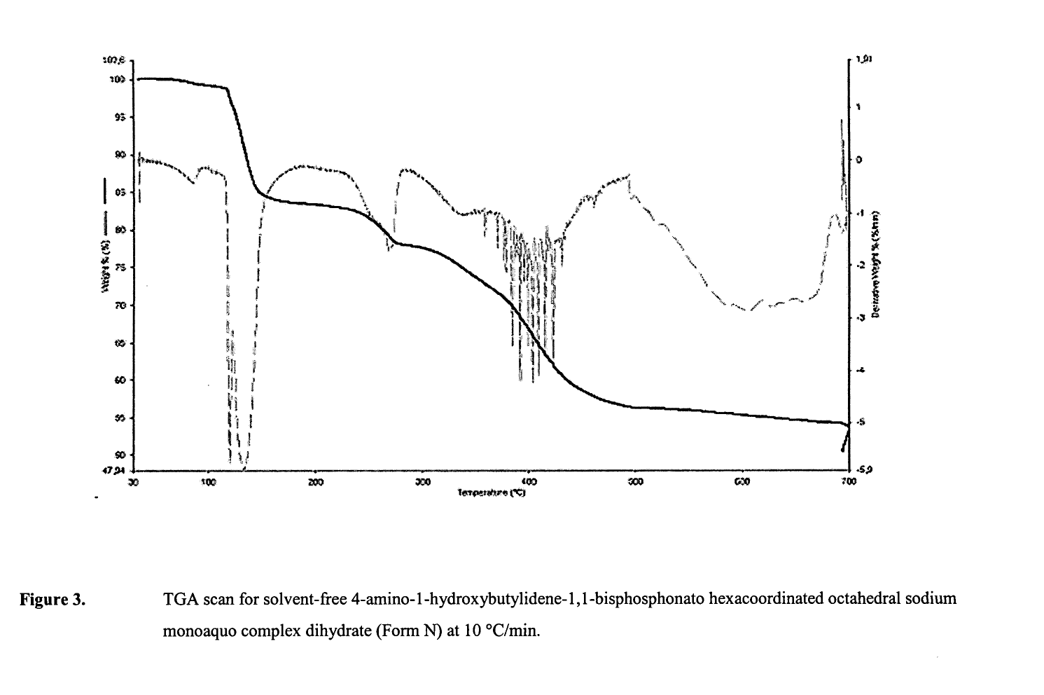

The TGA curve for solvent-free 4-amino-1-hydroxybutylidene-1,1-bisphosphonato hexacoordinated octahedral sodium monoaquo complex dihydrate (Form N), as shown in FIG. 3, exhibits three weight losses in the temperature region from 35° C. to 200° C. The first thermal event corresponds to loss of adsorbed water. The second and third thermal events correspond to loss of three water molecules, indicating that these three water molecules are bonded in a different manner within the crystal structure, e.g. one coordinated and two crystal water molecules, consequently needing different amounts of energy for dehydration. After 200° C., a series of weight losses occur corresponding to the degradation of Form N.

TABLE 1
Basic crystallographic data for the new solid-state form of solvent-free
4-amino-1-hydroxybutylidene-1,1-bisphosphonato hexacoordinated
octahedral sodium monoaquo complex dihydrate (Form N).

| | Form N |
|---|---|
| Empirical formula | [Na(C₄H₁₂NO₇P₂)₂(OH₂)].2 H₂O |
| Formula weight | 591.26 |
| Temperature | 100 K |
| Crystal size | 0.10 × 0.30 × 0.35 |
| Crystal system, space group | Monoclinic, P 2₁/n |
| Unit cell dimensions | a = 7.26(1) Å |
| | b = 8.95(1) Å |
| | c = 19.40(1) Å |
| | β = 100.3(1)° |
| | α = γ = 90° |
| Volume | 1240 Å³ |
| Z | 4 |
| Calculated density | 1.74(1) g cm⁻³ |

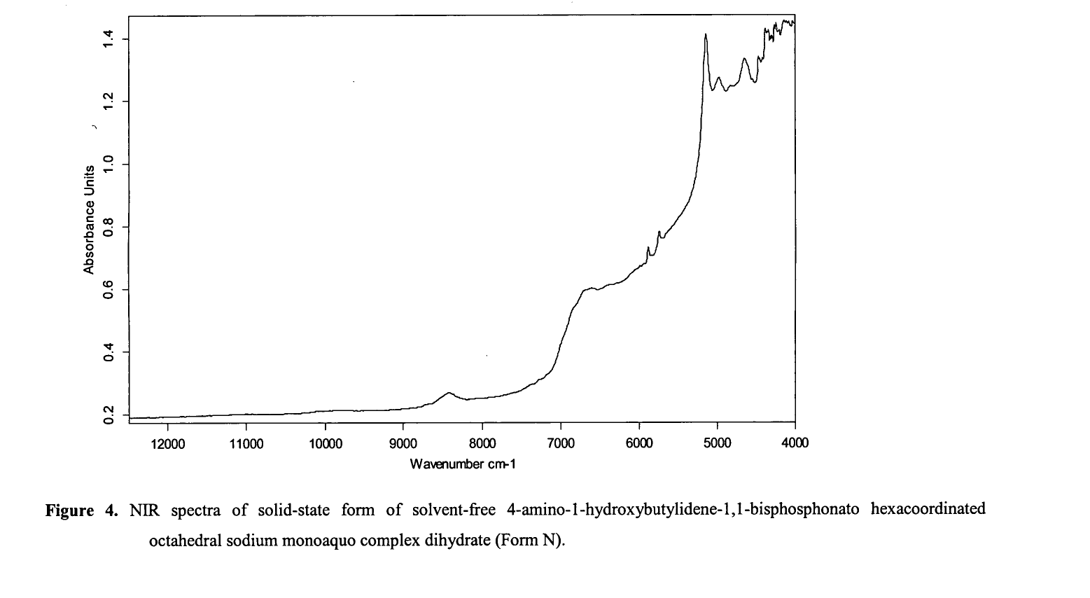

The characteristic near infra-red (NIR) spectrum for the solvent-free 4-amino-1-hydroxybutylidene-1,1-bisphosphonato hexacoordinated octahedral sodium monoaquo complex dihydrate (Form N) of the present invention is given in FIG. 4. The NIR spectrum was measured by Bruker NIR Multi Purpose Analyser (MPA) with He—Ne laser as a light source. The spectrum was obtained using a reflection fiber-optical solid probe. The optical probe tip was in close contact to the samples. The measurements were carried out in triplicate over the range 4000 cm⁻¹-12000 cm⁻¹ with resolution of 8 cm⁻¹ and the spectra were averaged over 32 scans. The new solid-state form of solvent-free 4-amino-1-hydroxybutylidene-1,1-bisphosphonato hexacoordinated octahedral sodium monoaquo complex dihydrate (Form N) has characteristic NIR peaks as follows: 5144±8, 4979±8, and 4649±8 cm⁻¹.

The solvent-free 4-amino-1-hydroxybutylidene-1,1-bisphosphonato hexacoordinated octahedral sodium monoaquo complex dihydrate (Form N) exhibits both chemical and physical stability, as confirmed after six months stability testing at 40° C./75% relative humidity (RH), 12 months at 30° C./60% RH, and after photostability testing.

Preparation of the New Solid-State Form of 4-amino-1-hydroxybutylidene-1,1-bisphosphonato Hexacoordinated Octahedral Sodium Monoaquo Complex Dihydrate (Form N)

The new solid-state Form N can be obtained by a process that involves reacting 4-amino-1-hydroxybutylidene-1,1-bisphosphonic acid with a strong acid cation exchange resin. In one embodiment, a solution of 4-amino-1-hydroxybutylidene-1,1-bisphosphonic acid is reacted with an strong acid cation exchange resin in the form of its sodium salt. Suitable strong acid cation exchange resins for use in the process include, but are not limited to, sodium ion forms of sulfonated divinylbenzene styrene copolymers, such as, but not limited to, AMBERLITE SR1L Na and AMBERJET 1200 Na, both of which are available from Rohm and Haas Company (Philadelphia, Pa.). The chemical and thermal stability, good ion exchange kinetics, and high exchange capacity of these strong acid cation exchange resins make the process described herein very economical. Moreover, short exchange reaction times at room temperature help to prevent product degradation.

Because the strong acid cation exchange resins are practically insoluble in most popular organic solvents, the solution of 4-amino-1-hydroxybutylidene-1,1-bisphosphonic acid used in the process is typically an aqueous solution. 4-amino-1-hydroxybutylidene-1,1-bisphosphonic acid is slightly soluble in water. Therefore, typical aqueous solution concentrations of 4-amino-1-hydroxybutylidene-1,1-bisphosphonic acid used in the process include, but are not limited to, from about 0.2% to about 1.5% by weight, preferably from about 0.5% to about 1% by weight, and more preferably about 1% by weight.

The exchange reaction is typically carried out at temperatures ranging from, but not limited to, about 20° C. to 80° C., for example, at about 70° C.

In one embodiment, the process is performed by flowing a solution of 4-amino-1-hydroxybutylidene-1,1-bisphosphonic acid through a column containing the strong acid cation exchange resin. Typical flow rates used in the exchange reaction include, but are not limited to, about 200 to about 700 cm³/hour, preferably from about 400 to about 500 cm³/hour. Typical exchange reaction times include, but are not limited to, about 3 hours to about 8 hours, preferably, from about 4 hours to about 5 hours.

The crude 4-amino-1-hydroxybutylidene-1,1-bisphosphonic acid monosodium, obtained by reaction of 4-amino-1-hydroxybutylidene-1,1-bisphosphonic acid with the strong acid cation exchange resin, can be crystallized from the concentrated eluate in about 85% yield and then recrystallized from water to yield 4-amino-1-hydroxybutylidene-1,1-bisphosphonato hexacoordinated octahedral sodium monoaquo complex dihydrate (Form N). Typical recrystallization temperatures include, but are not limited to from about 0° C. to about 10° C., preferably from about 0° C. to about 5° C.

Compositions of the New Solid-State Form N of Alendronate Sodium

The new solid-state form N of octahedral sodium monoaquo complex dihydrate complex of alendronate sodium of the present invention can be utilized in the preparation of rapid, controlled and sustained release pharmaceutical compositions, suitable for oral, parenteral, transdermal, buccal, or intravenous administration.

The compositions may be administered orally, in the form of rapid or controlled release tablets, microparticles, mini tablets, capsules, sachets, and oral solutions or suspensions, or powders for the preparation thereof. In addition to the new solid-state form N of octahedral sodium monoaquo complex dihydrate complex of alendronate sodium of the present invention as the active substance, oral preparations may optionally include various standard pharmaceutical carriers and excipients, such as binders, fillers, buffers, lubricants, glidants, dyes, disintegrants, odorants, sweeteners, surfactants, mold release agents, antiadhesive agents and coatings. Some excipients may have multiple roles in the compositions, e.g., act as both binders and disintegrants.

Examples of pharmaceutically acceptable disintegrants for oral compositions useful in the present invention include, but are not limited to, starch, pre-gelatinized starch, sodium starch glycolate, sodium carboxymethylcellulose, croscarmellose sodium, microcrystalline cellulose, alginates, resins, surfactants, effervescent compositions, aqueous aluminum silicates and crosslinked polyvinylpyrrolidone.

Examples of pharmaceutically acceptable binders for oral compositions useful herein include, but are not limited to, acacia; cellulose derivatives, such as methylcellulose, carboxymethylcellulose, hydroxypropylmethylcellulose, hydroxypropylcellulose or hydroxyethylcellulose; gelatin, glucose, dextrose, xylitol, polymethacrylates, polyvinylpyrrolidone, sorbitol, starch, pre-gelatinized starch, tragacanth, xanthane resin, alginates, magnesium-aluminum silicate, polyethylene glycol or bentonite.

Examples of pharmaceutically acceptable fillers for oral compositions include, but are not limited to, lactose, anhydrolactose, lactose monohydrate, sucrose, dextrose, mannitol, sorbitol, starch, cellulose (particularly microcrystalline cellulose), dihydro- or anhydro-calcium phosphate, calcium carbonate and calcium sulfate.

Examples of pharmaceutically acceptable lubricants useful in the compositions of the invention include, but are not limited to, magnesium stearate, talc, polyethylene glycol, polymers of ethylene oxide, sodium lauryl sulfate, magnesium lauryl sulfate, sodium oleate, sodium stearyl fumarate, DL-leucine and colloidal silicon dioxide Examples of suitable pharmaceutically acceptable odorants for the oral compositions include, but are not limited to, synthetic aromas and natural aromatic oils such as extracts of oils, flowers, fruits and combinations thereof. Examples are vanilla and fruit aromas, including banana, apple, sour cherry, peach and similar aromas. Their use depends on many factors, the most important being the organoleptic acceptability for the population that will be taking the pharmaceutical compositions.

Examples of suitable pharmaceutically acceptable dyes for the oral compositions include, but are not limited to, synthetic and natural dyes such as titanium dioxide, beta-carotene and extracts of grapefruit peel.

Examples of useful pharmaceutically acceptable coatings for the oral compositions, typically used to facilitate swallowing, modify the release properties, improve the appearance, and/or mask the taste of the compositions include, but are not limited to, hydroxypropylmethylcellulose, hydroxypropylcellulose and acrylate-methacrylate copolymers.

Suitable examples of pharmaceutically acceptable sweeteners for the oral compositions include, but are not limited to, aspartame, saccharin, saccharin sodium, sodium cyclamate, xylitol, mannitol, sorbitol, lactose and sucrose.

Suitable examples of pharmaceutically acceptable buffers include, but are not limited to, citric acid, sodium citrate, sodium bicarbonate, dibasic sodium phosphate, magnesium oxide, calcium carbonate and magnesium hydroxide.

Suitable examples of pharmaceutically acceptable surfactants include, but are not limited to, sodium lauryl sulfate and polysorbates.

Compositions of the solid-state form N of alendronate sodium of the present invention can also be administered intravenously or intraperitoneally, by infusion or injection. Dispersions can also be prepared in a liquid carrier or intermediate, such as glycerin, liquid polyethylene glycols, triacetin oils, and mixtures thereof. To improve storage stability, such preparations may also contain a preservative to prevent the growth of microorganisms.

Pharmaceutical compositions suitable for injection or infusion may be in the form of a sterile aqueous solution, a dispersion or a sterile powder that contains the active ingredient, adjusted, if necessary, for preparation of such a sterile solution or dispersion suitable for infusion or injection. This may optionally be encapsulated into liposomes. In all cases, the final preparation must be sterile, liquid, and stable under production and storage conditions.

The liquid carrier or intermediate can be a solvent or liquid dispersive medium that contains, for example, water, ethanol, a polyol (e.g. glycerol, propylene glycol or the like), vegetable oils, non-toxic glycerine esters and suitable mixtures thereof. Suitable flowability may be maintained, by generation of liposomes, administration of a suitable particle size in the case of dispersions, or by the addition of surfactants. Prevention of the action of micro-organisms can be achieved by the addition of various antibacterial and antifungal agents, e.g. paraben, chlorobutanol, or sorbic acid. In many cases isotonic substances are recommended, e.g. sugars, buffers and sodium chloride to assure osmotic pressure similar to those of body fluids, particularly blood. Prolonged absorption of such injectable mixtures can be achieved by introduction of absorption-delaying agents, such as aluminium monostearate or gelatin.

Sterile injectable solutions can be prepared by mixing the form N of alendronate sodium with an appropriate solvent and one or more of the aforementioned excipients, followed by sterile filtering. In the case of sterile powders suitable for use in the preparation of sterile injectable solutions, preferable preparation methods include drying in vacuum and lyophilization, which provide powdery mixtures of the isostructural pseudopolymorphs and desired excipients for subsequent preparation of sterile solutions.

The solid-state form N of alendronate sodium of the present invention may also be used for the preparation of locally acting, topical compositions. Such compositions may also contain other pharmaceutically acceptable excipients, such as polymers, oils, liquid carriers, surfactants, buffers, preservatives, stabilizers, antioxidants, moisturizers, emollients, colorants and odorants.

Examples of pharmaceutically acceptable polymers suitable for such topical compositions include, but are not limited to, acrylic polymers; cellulose derivatives, such as carboxymethylcellulose sodium, methylcellulose or hydroxypropylcellulose; natural polymers, such as alginates, tragacanth, pectin, xanthan and cytosan.

Examples of suitable pharmaceutically acceptable oils which are so useful include but are not limited to, mineral oils, silicone oils, fatty acids, alcohols, and glycols.

Examples of suitable pharmaceutically acceptable liquid carriers include, but are not limited to, water, alcohols or glycols such as ethanol, isopropanol, propylene glycol, hexylene glycol, glycerol and polyethylene glycol, or mixtures thereof in which the pseudopolymorph is dissolved or dispersed, optionally with the addition of non-toxic anionic, cationic or non-ionic surfactants, and inorganic or organic buffers.

Suitable examples of pharmaceutically acceptable preservatives include, but are not limited to, various antibacterial and antifungal agents such as solvents, for example ethanol, propylene glycol, benzyl alcohol, chlorobutanol, quaternary ammonium salts, and parabens (such as methyl paraben, ethyl paraben, propyl paraben, etc.).

Suitable examples of pharmaceutically acceptable stabilizers and antioxidants include, but are not limited to, ethylenediaminetetraacetic acid (EDTA), thiourea, tocopherol and butyl hydroxyanisole.

Suitable examples of pharmaceutically acceptable moisturizers include, but are not limited to, glycerine, sorbitol, urea and polyethylene glycol.

Suitable examples of pharmaceutically acceptable emollients include, but are not limited to, mineral oils, isopropyl myristate, and isopropyl palmitate.

The use of dyes and odorants in topical compositions of the present invention depends on many factors of which the most important is organoleptic acceptability to the population that will be using the pharmaceutical compositions.

The therapeutically acceptable quantity of the solid-state form N of octahedral sodium monoaquo complex dihydrate complex of alendronate sodium of the present invention administered varies, depending on the mode of administration, treatment conditions, age and status of the patient or animal species, and is subject to the final decision of the physician, clinician or veterinary doctor monitoring the course of treatment. For example, the solid-state form N may be formulated in a dosage form that contains from about 1 to about 350 mg of the active substance per unit dose, preferably from about 5 to about 150 mg of the active substance per unit dose.

The present invention also relates to methods of inhibiting bone resorption, treating or preventing osteoporosis and treating Paget's disease in a patient in need of such treatment by administering to the patient a pharmaceutical composition containing a therapeutically effective amount of new solid-state form N octahedral sodium monoaquo complex dihydrate complex of alendronate sodium of the present invention.

### EXAMPLES

The present invention is illustrated, but not limited by the following examples.

### Example 1

### Preparation with AMBERLITE SR1L Na Exchange Resin

(a) Preparation of the Cation Exchange Resin Column

300 mL of water was added to a vertically disposed glass column having an inner diameter of 24.2 mm. 64 g of AMBERLITE SR1L Na was placed in the column to generate the strong acid cation exchange resin bed. The resin bed was washed by flowing approximately 1000 ml of water through the column over a period of 20 minutes until the eluate was clear.

(b) Preparation of Solvent-Free 4-amino-1-hydroxybutylidene-1,1-bisphosphonato Hexacoordinated Octahedral Sodium Monoaquo Complex Dihydrate (Form N) Using AMBERLITE SR1L Na

4-amino-1-hydroxybutylidene-1,1-bisphosphonic acid (20 g, 0.08 mol) was dissolved with stirring in 2000 ml distilled water at 70° C. The resulting solution was loaded into an AMBERLITE SR1L Na resin column prepared as described in step (a) above and passed over the column at a flow rate of 400-500 cm³/h. No further heat was applied. The pH of the initial eluate was 4.8. The pH of the final eluate was 4.2, indicating complete elution of the 4-amino-1-hydroxybutylidene-1,1-bisphosphonic acid monosodium from the ion exchange resin bed. The total time for elution was between 4 and 5 hours. 200 mL of water was then passed through the column. The pH of the 2200 mL of total eluate was in the range of 4.3-4.5.

The eluate was concentrated to about 60-80 mL under reduced pressure. The resulting solution was cooled in an ice-water bath at 0-5° C. for 3-4 hours, and the precipitate of 4-amino-1-hydroxybutylidene-1,1-bisphosphonic acid monosodium that formed was filtered off. The yield after drying at 70° C. for 2 hours to a constant weight was 21.5 g (82.4%). The crude product was recrystallized from water to afford the final solvent-free 4-amino-1-hydroxybutylidene-1,1-bisphosphonato hexacoordinated octahedral sodium monoaquo complex dihydrate (Form N) in 89.2% yield.

The analysis of the reaction product of Example 1 is set forth in Table 2.

TABLE 2
Analysis of Reaction Product of Example 1.

| Test | Results |
|---|---|
| Appearance | White, crystalline powder |
| Assay (HPLC) | 99.61% |
| Water Content (Karl Fisher) | 17.19% |
| Loss on drying | 16.91% |
| pH of a 1.0% aqueous solution | 4.83 |

| Microchemical analysis: | Theoretical | Experimental |
|---|---|---|
| Carbon | 14.78% | 14.87% |
| Hydrogen | 5.58% | 5.64% |
| Nitrogen | 4.31% | 4.29% |

Mass spectrum m/z (M+H⁺): 272;

¹H NMR (D₂O, δ (ppm)): 2.71 (CH₂, t, 2), 1.69 (CH₂CH₂, m, 4).

¹H NMR, MS, XRPD and IR data of the solvent-free 4-amino-1-hydroxybutylidene-1,1-bisphosphonato hexacoordinated octahedral sodium monoaquo complex dihydrate (Form N) were consistent with its structure.

### Example 2

### Preparation With AMBERJET 1200 Na Exchange Resin

(a) Preparation of the Cationexchange Resin Column

300 mL of water was added to a vertically disposed glass column having an inner diameter of 24.2 mm. 75 g of AMBERJET 1200 Na was placed in the column to generate the strong acid cation exchange resin bed. The resin bed was washed by flowing approximately 1000 ml of water through the column over a period of 20 minutes until the eluate was clear.

(b) Preparation of Solvent-Free 4-amino-1-hydroxybutylidene-1-bisphosphonato Hexacoordinated Octahedral Sodium Monoaquo Complex Dihydrate (Form N) Using AMBERJET 1200 Na 4-amino-1-hydroxybutylidene-1,1-bisphosphonic acid (20 g, 0.08 mol) was dissolved with stirring in 2000 ml distilled water at 70° C. The resulting solution was loaded into an AMBERJET 1200 Na resin column prepared as described in step (a) above and passed over the column at a flow rate of 400-500 cm³/h. No further heat was applied. The pH of the initial eluate was 4.8. The pH of the final eluate was 4.2, indicating complete elution of the 4-amino-1-hydroxybutylidene-1,1-bisphosphonic acid monosodium from the ion exchange resin bed. The total time for elution was between 4 and 5 hours. 250 mL of water was then passed through the column. The pH of the 2000-2200 mL of total eluate was in the range 4.3-4.5.

The eluate was concentrated to about 60-80 mL under reduced pressure. The resulting solution was cooled in an ice-water bath at 0-5° C. for 3-4 hours, and the precipitate of 4-amino-1-hydroxybutylidene-1,1-bisphosphonic acid monosodium that formed was filtered off. The yield after drying at 70° C. for 2 hours to a constant weight was 22.1 g (84.7%). The crude product was recrystallized from water to afford the final solvent-free 4-amino-1-hydroxybutylidene-1,1-bisphosphonato hexacoordinated octahedral sodium monoaquo complex dihydrate (Form N) in 89.9% yield.

The analysis of the reaction product of Example 2 is set forth in Table 3

TABLE 3
Analysis of Reaction Product of Example 2

| Test | Results |
|---|---|
| Appearance | White, crystalline powder |
| Assay (HPLC) | 99.59% |
| Water Content (Karl Fisher) | 16.91% |
| Loss on drying | 16.62% |
| Heavy metals | <20 ppm |
| pH of a 1.0% aqueous solution | 4.82 |

| Microchemical analysis: | Theoretical | Experimental |
|---|---|---|
| Carbon | 14.78% | 14.87% |
| Hydrogen | 5.58% | 5.66% |
| Nitrogen | 4.31% | 4.24% |

### Example 3

Solid state Form N of alendronate sodium is mixed with pharmaceutically acceptable excipients, such as microcrystalline cellulose, lactose, croscarmellose sodium, and magnesium stearate, and tabletted to form tablets containing 6.5, 13.0, and 52.0 mg of Form N alendronate sodium (which is the molar equivalent of 5.0, 10.0, and 40.0 mg, respectively, of the free acid).

Tablets containing a therapeutically effective amount of solid state Form N alendronate sodium are administered to patients in need of inhibition of bone resorption, and/or in need of treatment for osteoporois and/or in need of treatment for Paget's disease.

The present invention is not to be limited in scope by the specific embodiments described herein. Various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference.

1. A solid-state form of alendronate sodium (Form N) comprising an octahedral sodium monoaquo complex dihydrate complex characterized by monoclinic space group P 2₁/n and unit cell parameters comprising: crystal axis lengths of a=7.26 (1) Å, b=8.95 (1) Å, c=19.40 (1) Å and angles between the crystal axes of a=γ=90° and β=100.3 (1)°.

2. The solid-state form of alendronate sodium according to claim 1, having characteristic x-ray powder diffraction peaks, designated by 2Θ and expressed in degrees at: 9.2±0.2°, 12.4±0.2°, 13.5±0.2°, 17.1±0.2°, 18.5±0.2°, 21.0±0.2°, 24.9±0.2°, 26.1±0.2° and 29.9±0.2°.

3. The solid-state form of alendronate sodium according to claim 1, having characteristic near infra-red peaks at 5144±8, 4979±8, and 4649±8 cm⁻¹.

4. A process for the preparation of alendronate sodium comprising
(a) reacting a solution of 4-amino-1-hydroxybutylidene-1,1-bisphosphonic acid with a strong acid cation exchange resin in the form of its sodium salt, thereby forming alendronate sodium.

5. The process of claim 4, further comprising (b) crystallizing the alendronate sodium.

6. The process of claim 4, wherein the exchange resin is a sodium ion form of a sulfonated divinylbenzene styrene copolymer.

7. The process according to claim 6 wherein the exchange resin is selected from AMBERJET 1200 Na and AMBERLITE SR1L Na.

8. The process of claim 4, wherein the solution comprises water.

9. The process of claim 4, wherein step (a) is conducted at about 20° C. to about 80° C.

10. The process of claim 4, where step (a) comprises flowing a solution of 4-amino-1-hydroxybutylidene-1,1-bisphosphonic acid through a column containing a strong acid cation exchange resin in the form of its sodium salt.

11. The process of claim 5 wherein step (b) is conducted at about 0° C. to about 5° C.

12. The solid state form of alendronate sodium of claim 1, prepared by a process comprising
(a) reacting a solution of 4-amino-1-hydroxybutylidene-1,1-bisphosphonic acid with a strong acid cation exchange resin in the form of its sodium salt, thereby forming alendronate sodium.

13. A pharmaceutical composition comprising a solid-state form of alendronate sodium of claim 1 and a pharmaceutically acceptable carrier.

14. A method for inhibiting bone resorption comprising administering to a patient in need thereof a therapeutically effective amount of a solid state form of alendronate sodium of claim 1.

15. A method for treating osteoporosis comprising administering to a patient in need thereof a therapeutically effective amount of a solid state form of alendronate sodium of claim 1.

16. A method for treating Paget's disease comprising administering to a patient in need thereof a therapeutically effective amount of a solid state form of alendronate sodium of claim 1.
